(19) United States
(12) Patent Application Publication
(10) Pub. No.: US 2026/0314713 A1
(43) Pub. Date: Oct. 15, 2026

(54) ELECTRONIC GARMENT

(71) Applicant: E Ink Holdings Inc., HSINCHU (TW)

(72) Inventors: Deng-Kuen SHIAU, HSINCHU (TW); Wei-Hung WENG, HSINCHU (TW); Zhaoyu YANG, HSINCHU (TW)

(21) Appl. No.: 19/438,674

(22) Filed: Jan. 2, 2026

(30) Foreign Application Priority Data
Apr. 10, 2025 (TW) 114113614

(51) Int. Cl. A41D 1/00; G09F 9/30

(52) U.S. Cl. CPC A41D 1/002; G09F 9/301

(57) ABSTRACT

An electronic garment includes a cloth carrier and a flexible display panel. The cloth carrier includes a base layer and an outer layer. The outer layer is disposed on the base layer and has an opening. The flexible display panel is disposed between the base layer and the outer layer, and has a display area for displaying images and a peripheral area adjacent to the display area. The opening exposes the display area, and the outer layer covers the peripheral area.

### CROSS-REFERENCE TO RELATED APPLICATION

This application claims priority to Taiwan Application Serial Number 114113614, filed Apr. 10, 2025, which is herein incorporated by reference in its entirety.

### BACKGROUND

### Field of Invention

The present disclosure relates to an electronic garment. More particularly, the present disclosure relates to an electronic garment including a flexible display panel.

### Description of Related Art

With the increasing development of the technology industry, display panels have become widely used in daily life. Combining display panels with cloth carriers to create electronic garments can enhance versatility and functionality by displaying different patterns. However, because display panels have borders that cannot display images, if the display panel is spliced with the cloth carrier in a general flat manner, an obvious gap will appear, or if the border of the display panel is folded and then spliced with the cloth carrier, uneven protrusions will appear, resulting in a decrease in aesthetics.

### SUMMARY

At least one embodiment of the present disclosure provides an electronic garment that can improve aesthetics.

The electronic garment according to at least one embodiment of the present disclosure includes a cloth carrier and a flexible display panel. The cloth carrier includes a base layer and an outer layer, and the outer layer is disposed on the base layer and has an opening. The flexible display panel is disposed between the base layer and the outer layer, and has a display area for displaying images and a peripheral area adjacent to the display area. The opening exposes the display area, and the outer layer covers the peripheral area.

The electronic garment according to at least another embodiment of the present disclosure includes a cloth carrier, a flexible display panel, a flexible circuit board and a driver circuit element. The cloth carrier includes a base layer, an outer layer and an additional layer. The outer layer is disposed on the base layer. The additional layer is connected to the base layer to form an accommodation space. The flexible display panel is disposed between the base layer and the outer layer, and has a display area for displaying images and a peripheral area adjacent to the display area. The outer layer covers the peripheral area. The flexible circuit board is electrically connected to the flexible display panel in the peripheral area. The driver circuit element is electrically connected to the flexible circuit board, and is used to drive the flexible display panel to display images. The driver circuit element is disposed in the accommodation space.

It is to be understood that both the foregoing general description and the following detailed description are by examples, and are intended to provide further explanation of the present disclosure as claimed.

### BRIEF DESCRIPTION OF THE DRAWINGS

The present disclosure can be more fully understood by reading the following detailed description of the embodiment, with reference made to the accompanying drawings as follows:

FIG. 1 is a schematic view of an electronic garment according to at least one embodiment of the present disclosure.

FIG. 2 is a schematic cross-sectional view taken along line a-a’ of FIG. 1.

FIG. 3 is a block diagram of an electronic garment and an external electronic device according to at least one embodiment of the present disclosure.

### DETAILED DESCRIPTION

The embodiments of the present disclosure are discussed in detail below. It will be appreciated, however, that the embodiments provide many applicable concepts which may be implemented in a wide variety of specific contexts. The discussed and disclosed embodiments are for illustrative purposes only and are not intended to limit the scope of patent applications in this case.

In the following description, in order to clearly present the technical features of the present disclosure, the dimensions of elements in the drawings will be enlarged in unequal proportions. Therefore, the description and explanation of the following embodiments are not limited to the sizes and shapes presented by the elements in the drawings, but should cover the sizes, shapes, and deviations of the two due to actual manufacturing processes and/or tolerances. For example, the flat surface shown in the drawings may have rough and/or non-linear characteristics, and the acute angle shown in the drawings may be round. Therefore, the elements presented in the drawings in this case are mainly for illustration, and are not intended to accurately depict the actual shape of the elements, nor are they intended to limit the scope of patent applications in this case.

The spatial relative terms used in the present disclosure, such as "below," "under," "above," "on," and the like, are intended to facilitate the recitation of a relative relationship between one element or feature and another as depicted in the drawings. The true meaning of these spatial relative terms includes other orientations. For example, the relationship between one element and another may change from "below" and "under" to "above" and "on" when the drawing is turned 180 degrees up or down. In addition, spatially relative descriptions used in the present disclosure should be interpreted in the same manner.

It should be understood that while the present disclosure may use terms such as "first", "second", "third" to describe various elements or features, these elements or features should not be limited by these terms. These terms are primarily used to distinguish one element from another or one feature from another. In addition, the term "or" as used in the present disclosure may include, as appropriate, any one or a combination of the listed items in association.

Moreover, the present disclosure may be implemented or applied in various other specific embodiments, and the details of the present disclosure may be combined, modified, and altered in various embodiments based on different viewpoints and applications, without departing from the idea of the present disclosure.

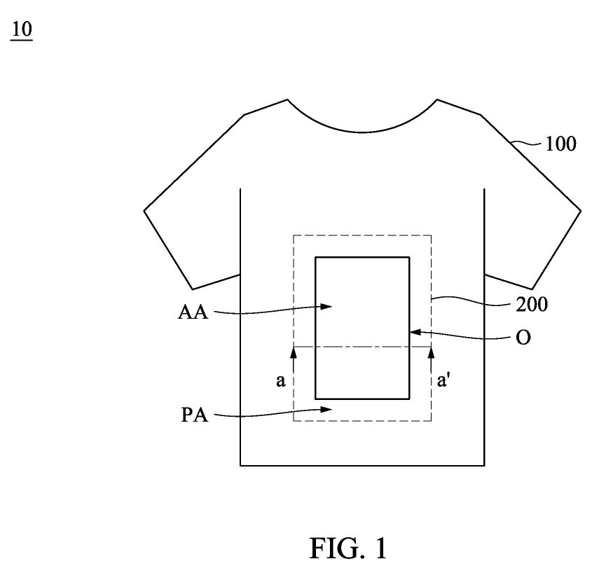

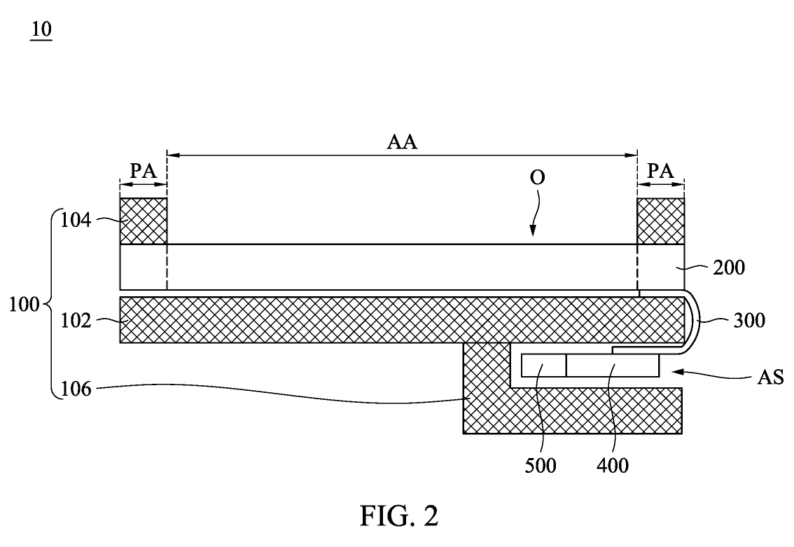

FIG. 1 is a schematic view of an electronic garment 10 according to at least one embodiment of the present disclosure. FIG. 2 is a schematic cross-sectional view taken along line a-a’ of FIG. 1. Referring to FIGS. 1 and 2, the electronic garment 10 includes a cloth carrier 100 and a flexible display panel 200. The cloth carrier 100 includes a base layer 102 and an outer layer 104. The outer layer 104 is disposed on the base layer 102 and has an opening O. The flexible display panel 200 is disposed between the base layer 102 and the outer layer 104, and has a display area AA for displaying images and a peripheral area PA adjacent to the display area AA. The opening O exposes the display area AA, and the outer layer 104 covers the peripheral area PA. In some embodiments, the size of the opening O is not larger than the size of the display area AA. The peripheral area PA surrounds the display area AA and can be used to arrange external wirings (not shown) of the flexible display panel 200. The flexible display panel 200 may include electronic paper, such as microcup electronic paper or microcapsule electronic paper. The material of the cloth carrier 100 may include woven cloth, non-woven cloth, plastic cloth, or any combination thereof.

Since the outer layer 104 of the cloth carrier 100 covers the peripheral area PA of the flexible display panel 200 and has the opening O that exposes the display area AA of the flexible display panel 200, there is no gap between the flexible display panel 200 and the cloth carrier 100. The flexible display panel 200 can smoothly form a seamless splicing with the cloth carrier 100 without bending the peripheral area PA, thereby improving the aesthetics of the electronic garment 10.

Referring to FIG. 2, the electronic garment 10 further includes a flexible circuit board 300 and a driver circuit element 400. The flexible circuit board 300 is electrically connected to the flexible display panel 200 in the peripheral area PA. The driver circuit element 400 is electrically connected to the flexible circuit board 300 and is used to drive the flexible display panel 200 to display images. The cloth carrier 100 further includes an additional layer 106. The additional layer 106 is connected to the base layer 102 to form an accommodation space AS, such as an inner pocket. The driver circuit element 400 is disposed in the accommodation space AS, and the flexible circuit board 300 is electrically connected to the driver circuit element 400 in the accommodation space AS. In addition, the electronic garment 10 further includes a battery 500. The battery 500 is disposed in the accommodation space AS to be electrically connected to the driver circuit element 400 to provide power to the flexible display panel 200.

Since the driver circuit element 400 is used to drive the flexible display panel 200 to display images, the versatility of the electronic garment 10 is increased. Moreover, the battery 500 is used to provide power to the flexible display panel 200. When the power is exhausted, another battery can be replaced without removing the electronic garment 10 from the user and then charging the electronic garment 10 with a plug, thereby improving the convenience of use.

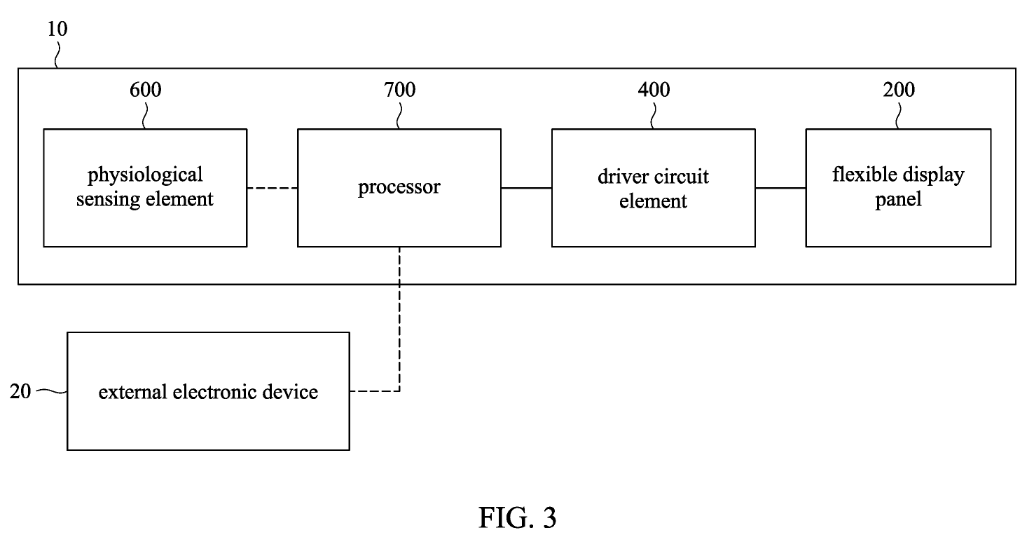

FIG. 3 is a block diagram of an electronic garment 10 and an external electronic device 20 according to at least one embodiment of the present disclosure. The electronic garment 10 further includes a physiological sensing element 600 and a processor 700. The physiological sensing element 600 is used to sense physiological values of a user (not shown) wearing the electronic garment 10. The processor 700 is electrically connected to the driver circuit element 400 for receiving conditions related to the physiological values set by the external electronic device 20 and receiving the physiological values, and sending instructions to the driver circuit element 400 according to the conditions to drive the flexible display panel 200 to display images.

In some embodiments, the physiological sensing element 600 and the processor 700 may be disposed in the accommodation space AS, but the present disclosure is not limited thereto. The external electronic device 20 may include a computer, a mobile phone, etc., and the processor 700 may receive the above-mentioned conditions and the above-mentioned physiological values via wireless transmission, such as a network. The physiological values may include heart rate, body temperature, blood sugar level, or any combination thereof.

In detail, the above-mentioned condition may include a default value, and the above-mentioned instruction may include displaying a first image and displaying a second image different from the first image. When the physiological value is greater than the default value, the flexible display panel 200 displays the first image; when the physiological value is less than the default value, the flexible display panel 200 displays the second image. For example, when the heart rate is greater than the default value, the processor 700 sends the instruction to the driver circuit element 400 to drive the flexible display panel 200 to display the first image; when the heart rate is less than the default value, the processor 700 sends the instruction to the driver circuit element 400 to drive the flexible display panel 200 to display the second image.

Alternatively, the above-mentioned condition may include a first default state and a second default state different from the first default state. The above-mentioned instruction may include displaying a first image and displaying a second image different from the first image. When the trend of the physiological value meets the first default state, the flexible display panel 200 displays the first image; when the trend of the physiological value meets the second default state, the flexible display panel 200 displays the second image. For example, when the heart rate increases, the processor 700 sends the instruction to the driver circuit element 400 to drive the flexible display panel 200 to display a dynamic image in which the color blocks scroll from bottom to top; when the heart rate decreases, the processor 700 sends the instruction to the driver circuit element 400 to drive the flexible display panel 200 to display a dynamic image in which the color blocks scroll from top to bottom. With the above-mentioned design, the electronic garment 10 can be applied in fields such as medical care and sports that require real-time detection and display of physiological status.

In summary, in at least one embodiment of the electronic garment of the present disclosure, the flexible display panel is disposed between the base layer and the outer layer of the cloth carrier, and the outer layer of the cloth carrier covers the peripheral area of the flexible display panel and has an opening exposing the display area of the flexible display panel. Therefore, there is no gap between the flexible display panel and the cloth carrier, and the flexible display panel can smoothly form a seamless splicing with the cloth carrier without bending the peripheral area, thereby improving the aesthetics of the electronic garment.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the present disclosure. In view of the foregoing, it is intended that the present disclosure cover modifications and variations of this disclosure provided they fall within the scope of the following claims.

What is claimed is:

1. An electronic garment, comprising:
a cloth carrier, comprising:
a base layer; and
an outer layer, disposed on the base layer and having an opening; and
a flexible display panel, disposed between the base layer and the outer layer, and having a display area for displaying images and a peripheral area adjacent to the display area, wherein the opening exposes the display area, and the outer layer covers the peripheral area.

2. The electronic garment of claim 1, wherein the flexible display panel comprises an electronic paper.

3. The electronic garment of claim 2, wherein the electronic paper comprises a microcup electronic paper or a microcapsule electronic paper.

4. The electronic garment of claim 1, wherein a size of the opening is not larger than a size of the display area.

5. The electronic garment of claim 1, further comprising:
a flexible circuit board, electrically connected to the flexible display panel in the peripheral area; and
a driver circuit element, electrically connected to the flexible circuit board, and used to drive the flexible display panel to display images.

6. The electronic garment of claim 5, wherein the cloth carrier further comprises:
an additional layer, connected to the base layer to form an accommodation space, wherein the driver circuit element is disposed in the accommodation space.

7. The electronic garment of claim 6, wherein the flexible circuit board is electrically connected to the driver circuit element in the accommodation space.

8. The electronic garment of claim 6, further comprising:
a battery, disposed in the accommodation space to be electrically connected to the driver circuit element.

9. The electronic garment of claim 5, further comprising:
a physiological sensing element, used to sense a physiological value of a user wearing the electronic garment; and
a processor, electrically connected to the driver circuit element, used to receive a condition related to the physiological value set by an external electronic device and receive the physiological value, and used to send an instruction to the driver circuit element in response to the condition to drive the flexible display panel to display images.

10. The electronic garment of claim 9, wherein the physiological value comprises heart rate, body temperature, blood sugar level or any combination thereof.

11. The electronic garment of claim 9, wherein the processor receives the condition and the physiological value via wireless transmission.

12. The electronic garment of claim 9, wherein the condition comprises a default value, and the instruction comprises displaying a first image and displaying a second image different from the first image, wherein when the physiological value is greater than the default value, the flexible display panel displays the first image; when the physiological value is less than the default value, the flexible display panel displays the second image.

13. The electronic garment of claim 9, wherein the condition comprises a first default state and a second default state different from the first default state, and the instruction comprises displaying a first image and displaying a second image different from the first image, wherein when a trend of the physiological value meets the first default state, the flexible display panel displays the first image; when the trend of the physiological value meets the second default state, the flexible display panel displays the second image.

14. The electronic garment of claim 1, wherein the cloth carrier comprises woven cloth, non-woven cloth, plastic cloth or any combination thereof.

15. An electronic garment, comprising:
a cloth carrier, comprising:
a base layer;
an outer layer, disposed on the base layer; and
an additional layer, connected to the base layer to form an accommodation space;
a flexible display panel, disposed between the base layer and the outer layer, and having a display area for displaying images and a peripheral area adjacent to the display area, wherein the outer layer covers the peripheral area;
a flexible circuit board, electrically connected to the flexible display panel in the peripheral area; and
a driver circuit element, electrically connected to the flexible circuit board, and used to drive the flexible display panel to display images, wherein the driver circuit element is disposed in the accommodation space.
